(19) United States
(12) Patent Application Publication
(10) Pub. No.: US 2004/0259115 A1
(43) Pub. Date: Dec. 23, 2004

(54) Methods and compositions for RNA detection and quantitation

(76) Inventors: David M. Schuster, Poolesville, MD (US); Ayoub Rashtchian, Gaithersburg, MD (US)

Correspondence Address: HELLER EHRMAN WHITE & MCAULIFFE LLP, 1666 K STREET,NW, SUITE 300, WASHINGTON, DC, 20006, US

(21) Appl. No.: 10/766,312

(22) Filed: Jan. 29, 2004

Related U.S. Application Data
(60) Provisional application No. 60/443,181, filed on Jan. 29, 2003.

(51) Int. Cl. C12Q 1/68; C12P 19/34

(52) U.S. Cl. 435/6; 435/91.2

(57) ABSTRACT

The present invention generally relates to methods of making cDNA molecules, amplification of RNA by PCR and cDNA libraries. The invention also relates to kits for carrying out the methods of the invention. Methods for improved and more efficient conversion of RNA into cDNA are provided, which in turn can be used in a variety of procedures in molecular analysis of gene expression.

The present invention is also directed to compositions comprising mixtures of reagents, including reverse transcriptases, buffers, cofactors and other components, suitable for immediate use in conversion of RNA into cDNA and RT PCR without dilution or addition of further components. These compositions are useful, alone or in the form of kits, for cDNA synthesis or nucleic acid amplification (e.g., by the Polymerase Chain Reaction) or for any procedure utilizing reverse transcriptases in a variety of research, medical, diagnostic, forensic and agricultural applications.

This application claims priority to application Ser. No. 60/443,181, filed Jan. 29, 2003, the contents of which are hereby incorporated by reference in their entirety.

### FIELD OF THE INVENTION

This invention is in the fields of molecular and cellular biology. The invention provides improved reagent compositions and methods for preparing cDNA.

### BACKGROUND OF THE INVENTION

The invention provides improved methods of synthesizing cDNA. More specifically, the present invention provides methods of increasing the efficiency of cDNA synthesis and, more particularly, for increasing the sensitivity and accuracy of quantification of gene expression. Thus, the present invention provides improved cDNA synthesis useful in gene discovery, genomic research, diagnostics and identification of and detection of differentially expressed genes and identification of genes of importance in disease processes.

In examining the structure and physiology of an organism, tissue or cell, it is often desirable to determine its genetic content. The genetic framework of an organism is encoded in the double-stranded sequence of nucleotide bases in the deoxyribonucleic acid (DNA) which is contained in the somatic and germ cells of the organism. The genetic content of a particular segment of DNA, or gene, is only manifested upon production of the protein which the gene encodes. In order to produce a protein, a complementary copy of one strand of the DNA double helix (the “coding” strand) is produced by polymerase enzymes, resulting in a specific sequence of ribonucleic acid (RNA). This particular type of RNA, since it contains the genetic message from the DNA for production of a protein, is called messenger RNA (mRNA).

Within a given cell, tissue or organism, there exist many mRNA species, each encoding a separate and specific protein. This fact provides a powerful tool to investigators interested in studying genetic expression in a tissue or cell. mRNA molecules may be isolated and further manipulated by various molecular biological techniques, thereby allowing the elucidation of the full functional genetic content of a cell, tissue or organism. The identity and levels of specific mRNAs present in a particular sample provides clues to the biology of the particular tissue or sample being studied. Therefore, the detection, analysis, transcription, and amplification of RNAs are among the most important procedures in modern molecular biology.

A common approach to the study of gene expression is the production of complementary DNA (cDNA). In this technique, the mRNA molecules from an organism are isolated from an extract of the cells or tissues of the organism. From these purified mRNA molecules, cDNA copies may be made using the enzyme reverse transcriptase (RT) or DNA polymerases having RT activity, which results in the production of single-stranded cDNA molecules. The term “reverse transcriptase” describes a class of polymerases characterized as RNA dependent DNA polymerases. All known reverse transcriptases require a primer to synthesize a DNA transcript from an RNA template.

Avian myoblastosis virus (AMV) reverse transcriptase was the first widely used RNA dependent DNA polymerase (Verma, Biochem. Biophys. Acta 473:1(1977)). The enzyme has 5′-3′ RNA directed DNA polymerase activity, 5′-3′ DNA directed DNA polymerase activity, and RNase H activity. RNase H is a processive 5′ and 3′ ribonuclease specific for the RNA strand for RNA DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNase H activity has been presented by Berger et al., Biochemistry 22:2365 2372 (1983).

Another reverse transcriptase which is used extensively in molecular biology is reverse transcriptase originating from Moloney murine leukemia virus (M-MLV). See, e.g., Gerard, G. R., DNA 5:271 279 (1986) and Kotewicz, M. L., et al., Gene 35:249 258 (1985). M-MLV reverse transcriptase substantially lacking in RNase H activity has also been described. See, e.g., U.S. Pat. No. 5,244,797.

Historically, reverse transcriptase has been used primarily to transcribe mRNA into cDNA which can then be cloned into a vector for further manipulation. The single-stranded cDNAs may be converted into a complete double-stranded DNA copy (i.e., a double-stranded cDNA) of the original mRNA (and thus of the original double-stranded DNA sequence, encoding this mRNA, contained in the genome of the organism) by the action of a DNA polymerase. The double stranded cDNAs can then be inserted into a vector, transformed into an appropriate bacterial, yeast, animal or plant cell host, and propagated as a population of host cells containing a collection of cDNA clones, or cDNA library, that represents the genes, or portions of genes present in the original mRNA sample.

Alternatively, cDNA can be labeled with an appropriate reporter moiety and used as hybridization probe to query defined target sequences immobilized on glass slides, filters, or other suitable solid supports. The identity and relative abundance of a given mRNA in a sample can be inferred from the signal intensity for a specific target sequence on the solid support.

One of the most widely used techniques to study gene expression exploits first-strand cDNA for mRNA sequence(s) as template for amplification by the polymerase chain reaction, PCR. This method, often referred to as RNA PCR or reverse transcriptase PCR (RT-PCR), exploits the high sensitivity and specificity of the PCR process and is widely used for detection and quantification of RNA. Recently, the ability to measure the kinetics of a PCR reaction by on-line detection in combination with these RT-PCR techniques has enabled accurate and precise measurement of RNA sequences with high sensitivity. This has become possible by detecting the RT-PCR product through fluorescence monitoring and measurement of PCR product during the amplification process by fluorescent dual-labeled hybridization probe technologies, such as the “TaqMan” 5′ fluorogenic nuclease assay described by Holland et al. (Proc. Natl. Acad. Sci. U.S.A. 88, 7276 (1991)), Gibson et al. (Genome Res. 6, 99 (1996)), and Heid et al. (Genome Res. 6, 986 (1996)); or “Molecular Beacons” (Tyagi, S. and Kramer, F. R. Nature Biotechnology 14, 303 (1996)). Nazarenko et al. (Nucleic. Acids Res. 25, 2516 (1997)) have described use of dual-labeled hairpin primers, as well as recent modifications utilizing primers labeled with only a single fluorophore (Nazerenko et al., Nucleic. Acids Res. (2002)). One of the more widely used methods is the addition of double-strand DNA-specific fluorescent dyes to the reaction such as: ethidium bromide (Higuchi et al., Biotechnology (1992) and Higuchi et al., Biotechnology 11, 102610, 413 (1993)), YO-PRO-1 (Ishiguro et al., Anal. Biochem. 229, 207 (1995)), or SYBR Green I (Wittwer et al., Biotechniques 22,130 (1997)). These improvements in the PCR method have enabled simultaneous amplification and homogeneous detection of the amplified nucleic acid without purification of PCR product or separation by gel electrophoresis. This combined approach decreases sample handling, saves time, and greatly reduces the risk of product contamination for subsequent reactions, as there is no need to remove the samples from their closed containers for further analysis. The concept of combining amplification with product analysis has become known as “real time” PCR, also referred to as quantitative PCR, or qPCR.

The general principals for template quantification by real-time PCR were first disclosed by Higuchi R, G Dollinger, P S Walsh and R. Griffith, “Simultaneous amplification and detection of specific DNA sequences”, Bio/Technology 10:413-417, 1992; Higuchi R, C Fockler G Dollinger and R Watson, Kinetic PCR analysis: real time monitoring of DNA amplification reactions, Bio/Technology 11:1026-1030. This simpler approach for quantitative PCR utilizes a double-strand specific fluorescent dye, ethidium bromide, added to amplification reaction. The fluorescent signal generated at each cycle of PCR is proportional to the amount of PCR product. A plot of fluorescence versus cycle number is used to describe the kinetics of amplification and a fluorescence threshold level was used to define a fractional cycle number related to initial template concentration. Specifically, the log of the initial template concentration is inversely proportional to the fractional cycle number (threshold cycle, or Ct), defined as the intersection of the fluorescence versus cycle number curve with the fluorescence threshold. Higher amounts of starting template results in PCR detection at a lower Ct value, whereas lower amounts require a greater number of PCR cycles to achieve an equivalent fluorescent threshold (Ct) and are detected at higher Ct values. Typically, the setting of this fluorescence threshold is defined as a level that represents a statistically significant increase over background fluorescent noise. Since this occurs at an early stage in the PCR process when critical substrates are not limiting, quantification of starting template occurs over a broad dynamic range with high accuracy, precision, and sensitivity. A major problem in understanding of gene expression patterns for gene discovery and identification of metabolic pathways is the limitations of current methods for accurate quantification. Use of real time PCR methods provides a significant improvement towards this goal. However, real-time PCR quantification of mRNA is still bounded by limitations of the process of reverse transcription.

The RT-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with RT-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” RT-PCR procedure (e.g., two step RT-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” RT PCR methods use a common or compromised buffer for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn⁺⁺ then PCR is carried out in the presence of Mg⁺⁺ after the removal of Mn⁺⁺ by a chelating agent. Finally, the “continuous” method (e.g., one step RT-PCR) integrates the three RT-PCR steps into a single continuous reaction that avoids the opening of the reaction tube for component or enzyme addition. Continuous RT-PCR has been described as a single enzyme system using the reverse transcriptase activity of thermostable Taq DNA Polymerase and Tth polymerase and as a two enzyme system using AMV RT and Taq DNA Polymerase wherein the initial 65° C. RNA denaturation step was omitted.

One step RT-PCR provides several advantages over uncoupled RT-PCR. One step RT-PCR requires less handling of the reaction mixture reagents and nucleic acid products than uncoupled RT-PCR (e.g., opening of the reaction tube for component or enzyme addition in between the two reaction steps), and is therefore less labor intensive, reducing the required number of person hours. One step RT-PCR also requires less sample, and reduces the risk of contamination (Sellner and Turbett, 1998). The sensitivity and specificity of one-step RT-PCR has proven well suited for studying expression levels of one to several genes in a given sample or the detection of pathogen RNA. Typically, this procedure has been limited to use of gene-specific primers to initiate cDNA synthesis.

In one step RT PCR, depending on the reverse transcriptase used specific conditions have been developed so that the conditions are supportive of cDNA synthesis by RT and subsequent PCR amplification with a thermostable DNA polymerase such as Taq. The conditions described are specific for the reverse transcriptase of choice. For example Lee and Rashtchian have described use of MMLV reverse transcriptase in conjunction with buffers containing MgSO4. While Selner et al have described use AMV type enzymes in sulfate-containing buffers for one-step RT-PCR.

Attempts to streamline the process of RT-PCR have not been easy, and several reports have documented an interference between reverse transcriptase and thermostable DNA polymerase Taq when used in combination in a single tube RT-PCR resulting in low sensitivity or lack of results. For example, there has been at least one report of a general inhibition of Taq DNA polymerase when mixed with reverse transcriptases in one-step/one tube RT-PCR mixtures (Sellner, L. N., et al., Nucl. Acids Res. 20(7):1487-1490 (1992)). This same report indicated that the inhibition was not limited to one type of RT: both AMV-RT and M-MLV-RT inhibited Taq DNA polymerase and limited the sensitivity of RT-PCR. Under the reaction conditions used in the Sellner et al. studies (67 mM Tris-HCl, pH 8.8, 17 mM (NH₄)₂SO₄, 1 mM .beta.-mercaptoethanol, 6 μM EDTA, 0.2 mg/ml gelatin), the degree of Taq polymerase inhibition was found to increase with increasing RT concentration, up to a ratio of approximately 3 units of RT:2 units of Taq DNA polymerase beyond which Taq polymerase was rendered completely inactive.

Other reports describe attempts to develop conditions for one-step RT-PCR reactions. For example, the use of AMV-RT for one-step RT-PCR in a buffer comprising 10 mM Tris-HCl, (pH 8.3), 50 mM KCl, 1.5 mM MgCl₂, and 0.01% gelatin has been reported (Aatsinki, J. T., et al., BioTechniques 16(2):282-288 (1994)), while another report demonstrated one-step RT-PCR using a composition comprising AMV-RT and Taq DNA polymerase in a buffer consisting of 10 mM Tris-HCl, pH 8.3, 50 mM KCl, 0.01% gelatin and 1.5 mM MgCl₂ (Mallet, F., et al., BioTechniques 18(4):678-687 (1995)). Under the reaction conditions used in the latter report, substitution of M-MLV-RT (RNase H⁺ or RNase H⁻ forms) for AMV-RT showed the same activity in the continuous RT-PCR reaction.

It is known that different reverse transcriptases vary in their preference for optimal conditions and also vary with regard to their ability to copy different RNA's. Therefore a variety of engineered reverse transcriptases have been developed that provide improved reverse transcription. Despite these improvements, effective and complete reverse transcription of RNA remains a problem. Therefore, some researchers prefer to use MMLV type enzymes, while others prefer AMV type enzymes. An ideal solution would be to produce an enzyme that overcomes the deficiencies of both enzymes and that produces cDNA that is not biased with respect either to sequence, RNA secondary structure differences or to inefficiency of a particular reverse transcriptase to copy some sequences.

To this end Gerard et al (U.S. Patent Application 2002/0081581 A1) and Legerski (U.S. Pat. No. 6,406,891) have described the use of multiple reverse transcriptases to improve the yield and percentage of full-length cDNA during the construction of cDNA libraries. The underlying rationale for using such mixtures is that different RTs pause and terminate reverse transcription at different sequences. It is speculated that the point at which one RT pauses is further extended by the activity of the other polymerase in the reaction. However, examination of the data reported by Gerard et al. shows that not all combinations of reverse trancriptases or polymerases produced the desired effect. In general, improved cDNA synthesis was limited to mixtures of RNase H deficient RSV RT and RNase H(−) MMLV. In fact, combinations of an RNase H(+) RT with an RNase H(−) RT produced lower cDNA yields and less full-length product than that obtained with the RNase deficient RT alone. While the inventors suggested application of multiple RTs to methods for nucleic acid amplification, neither Gerard et al., nor Lergerski describe any reaction conditions that are compatible with coupled or one-step RT-PCR procedures. Furthermore, since the one-step RT-PCR is typically limited to short sequence fragments, improvements that increase the length of cDNA synthesis may not necessarily improve this process. It is known in the literature that reverse transcriptase in the one-step RT-PCR can result in inhibition of PCR. This result is dependent on the concentration and molecular properties of the reverse transcriptase. Based on this phenomenon, it is possible that mixtures of reverse transcriptases may exacerbate inhibition of amplification. Compatibility of a reverse transcriptase mixture with a given DNA polymerase is also likely to be dependent on the properties of the specific reverse transcriptases in the mix. One commercial manufacturer produces several one-step RT-PCR kits that employ a mixture of Sensiscript™ and OmniScript™, two RNase H (+) heterodimeric RTs. However, this combination of reverse transcriptases is prone to sequence bias and therefore limits accurate quantification of different mRNA species. Hence, there exists a need for one-step RT-PCR methods that overcome sequence bias and provides accurate quantification of RNAs independent of the amplified target sequence.

### SUMMARY OF THE INVENTION

It is therefore a goal of the present invention to provide mixtures containing at least two reverse transcriptases that work synergistically under the same buffer conditions to produce more complete cDNAs that are useful for expression profiling.

It is a further goal of the invention to provide improved buffer compositions that support different reverse transcriptases and that are suitable for 1-step RT PCR. Use of a mixture of RT enzymes in 1-step RT PCR results in superior results and accurate quantitation of different mRNA's in complex samples.

In accordance with these goals, there is provided in one aspect of the invention, a method for amplifying a nucleic acid molecule, including the step of incubating an RNA template with a composition comprising (a) a buffer, (b) two or more proteins having reverse transcriptase activity and (c) at least one DNA polymerase under conditions which substantially relieve reverse-transcriptase-mediated inhibition of DNA polymerase activity and which are sufficient to amplify a DNA molecule complementary to all or a portion of said RNA template.

In one embodiment, the two or more proteins having reverse transcriptase activity may be a first reverse transcriptase enzyme in which the reverse transcriptase activity resides in a single polypeptide and a second reverse transcriptase enzyme in which the reverse transcriptase activity resides in a dimeric or multimeric structure.

In another embodiment of the invention, the first reverse transcriptase enzyme in which the reverse transcriptase activity resides in a single polypeptide may be a Moloney murine leukemia virus (M-MLV) reverse transcriptase or a derivative thereof having reduced RNAse H activity and the second reverse transcriptase enzyme in which the reverse transcriptase activity resides in a dimeric or multimeric structure may be an AMV reverse transcriptase or a derivative thereof having reduced RNAse H activity.

In any of these methods, the composition may contain a first primer and a second primer, where the first primer is suitable for facilitating synthesis of first strand cDNA from said RNA template, and where the combination of the first and said second primer is suitable for amplifying the first strand cDNA.

In the foregoing methods, the buffer may contain glutamate-containing molecules that aid in an amount that is effective to substantially relieve reverse-transcriptase-mediated inhibition of DNA polymerase activity. The concentration of the glutamate-containing molecules may be, for example, about 1 mM to about 500 mM.

In accordance with another aspect of the invention there has been provided a method for accurately quantifying a nucleic acid molecule in an essentially sequence-independent manner, comprising the steps of incubating an RNA template with a composition comprising (a) a buffer, (b) two or more proteins having reverse transcriptase activity, (c) at least one DNA polymerase, and (d) a first primer and a second primer, where the first primer is suitable for facilitating synthesis of first strand cDNA from the RNA template, and where the combination of the first and the second primer is suitable for amplifying the first strand cDNA, and where the said incubation is under conditions which substantially relieve reverse-transcriptase-mediated inhibition of DNA polymerase activity and which are sufficient to amplify a DNA molecule complementary to all or a portion of the RNA template.

In accordance with another aspect of the invention there has been provided a method for the unbiased quantification of a nucleic acid molecule contained in a sample, the method comprising incubating an RNA template with a composition comprising (a) a buffer, (b) two or more proteins having reverse transcriptase activity, (c) at least one DNA polymerase, and (d) a first primer and a second primer, where the first primer is suitable for facilitating synthesis of first strand cDNA from said RNA template, and where the combination of the first and the second primer is suitable for amplifying the first strand cDNA, and where the incubation is under conditions which substantially relieve reverse-transcriptase-mediated inhibition of DNA polymerase activity and which are sufficient to amplify a DNA molecule complementary to all or a portion of the RNA template.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The present invention provides compositions and methods useful for one-step/one-tube RT-PCR, preferably using two or more reverse transcriptases, or RNase H-deficient (“RNase H⁻”) derivatives thereof, in combination with one or more DNA polymerases. This invention also provides compositions and methods useful for one-step/one tube RT-PCR in the presence of buffer compositions (containing for example glutamate-containing molecules) that relieve the inhibition of PCR often observed when using compositions comprising two or more enzymes having reverse transcriptase activity, and allow PCR to proceed efficiently.

In particular, the invention is directed to methods for amplifying a nucleic acid molecule comprising (a) mixing an RNA template with a composition comprising a first reverse transcriptase (for example a Moloney murine leukemia virus (M-MLV) reverse transcriptase) and a second reverse transcriptase (for example an Avian myoblastosis virus reverse transcriptase), in combination with one or more DNA polymerases and (b) incubating the mixture under conditions sufficient to amplify a DNA molecule complementary to all or a portion of the RNA template. In particular the invention contemplates use of reverse transcriptase mixtures containing a single polypeptide reverse transcriptase enzyme (for example M-MLV) and a reverse transcriptase enzyme containing two or more subunits, for example AMV or the HIV reverse transcriptase).

In a related aspect, the invention provides buffer compositions for use in RT-PCR methods that contain one or more components that suppress the inhibition of polymerase activity by the presence of one or more reverse transcriptase. Thus, for example, the present inventors have discovered that the presence of glutamate is effective for suppressing RT-mediated inhibition of polymerase activity. In particular, one or more glutamate-containing molecules, such as one or more glutamate-containing buffers, may be used in step (a) of the above-described methods, wherein the concentration of the one or more glutamate-containing molecules is about 1 mM to about 500 mM. In one embodiment of such methods, at least one DNA-directed DNA polymerase is used, for example, one, two, or three polymerases may be used, where one or more of the polymerases is a thermostable DNA polymerases. Suitable polymerases include, but are not limited to, Thr, Tru, Tli, Tac, Tih, Tfi, Kod, Bst, Sac, Sso, Poc, Pab, Mth, Pho, ES4, VENT® (a variant DNA polymerase isolated from Thermococcus litoralis), and DEEPVENT® (a variant DNA polymerase isolated from Pyrococcus sp.), Tne, Tma, Taq, Pfu, Tth, Pwo, and Tfl, and mutants, variants and derivatives thereof.

In other aspects of the invention, the DNA polymerases may comprise a first DNA polymerase having 3′ exonuclease activity, most preferably a DNA polymerase selected from the group consisting of Pfu, Pwo, DEEPVENT, VENT, Tne, Tma, Kod, and mutants, variants and derivatives thereof, and a second DNA polymerase having substantially reduced 3′ exonuclease activity, for example a DNA polymerase selected from the group consisting of Taq, Tfl, Tth, and mutants, variants and derivatives thereof. In other embodiments the reverse transcriptases used can be at least two of the following enzymes: Moloney Murine Leukemia Virus (M-MLV) RT, Avian Myeloblastosis Virus (AMV) RT, Thermoscript, RSV, Enhanced Avian RT, Sensiscript, OmniScript, Superscript I, Superscript II, Superscript III, Tth DNA polymerase, Human Immunodeficiency Virus (HIV), Avian Sarcoma-Leukosis Virus (ASLV) RT, Rous Sarcoma Virus (RSV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus UR2AV RT, Avian Sarcoma Virus Y73 Helper Virus YAV RT, Rous Associated Virus (RAV) RT, and Myeloblastosis Associated Virus (MAV) RT.

Ideally, synthesis of a cDNA molecule initiates at a selected priming site on an mRNA molecule and terminates at the mRNA 5 ′-end, thereby generating cDNA which quantitatively represents the mRNA being studied. However, due to many factors such as inefficient priming, RNA length, nucleotide sequence composition, mRNA secondary structure and the inadequate processivity of reverse transcriptases, cDNA synthesis typically terminates prematurely, resulting in non-quantitative representation of different regions of mRNA (i.e. 3′-end sequences or 5′-end sequences). It has been demonstrated that use of mutant reverse transcriptases lacking RNase H activity can result in longer cDNA synthesis and better representation, and higher sensitivity of detection.

In studies involving quantitative analysis of gene expression, sequence bias in the cDNA and non-quantitative representation of different parts of mRNA can yield inaccurate expression data. Due to these problems a variety of reverse transcriptases and various mutants such as RNase H− mutants (Superscript II®, Thermoscript®) have been developed. Use of RNase H⁻ RT has been shown to result in cDNA yields that were approximately 4 fold higher than those obtained with M-MLV RT. A number of reports have attempted to develop a system for one-tube/one-step RT PCR. However, these attempts generally have been unsuccessful, and several reports have documented an interference between reverse transcriptase and thermostable Taq DNA polymerase when used in combination in a single tube RT-PCR, resulting in low sensitivity or lack of results.

For example, general inhibition of Taq DNA polymerase was observed when mixed with reverse transcriptases in one-step/one tube RT-PCR mixtures (Sellner, L. N., et al., Nucl. Acids Res. 20(7): 1487-1490 (1992)). This same report indicated that the inhibition was not limited to one type of RT: both AMV-RT and M-MLV-RT profoundly inhibited Taq DNA polymerase and limited the sensitivity of RT-PCR. Under the reaction conditions used in the Sellner et al. studies (67 mM Tris-HCl, pH 8.8, 17 mM (NH₄)₂SO₄, 1 mM β-mercaptoethanol, 6 μM EDTA, 0.2 mg/ml gelatin), the degree of Taq polymerase inhibition was found to increase with increasing RT concentration, up to a ratio of approximately 3 units of RT:2 units of Taq DNA polymerase, beyond which Taq polymerase was rendered completely inactive.

Other reports describe attempts to develop conditions for one-step RT-PCR reactions. For example, the use of AMV-RT for one-step RT-PCR in a buffer comprising 10 mM Tris-HCl, (pH 8.3), 50 mM KCl, 1.5 mM MgCl₂, and 0.01% gelatin has been reported (Aatsinki, J. T., et al., BioTechniques 16(2):282-288 (1994)), while another report demonstrated one-step RT-PCR using a composition comprising AMV-RT and Taq DNA polymerase in a buffer consisting of 10 mM Tris-HCl, pH 8.3, 50 mM KCl, 0.01% gelatin and 1.5 mM MgCl₂ (Mallet, F., et al., BioTechniques 18(4):678-687 (1995)). Under the reaction conditions used in the latter report, substitution of M-MLV-RT (RNase H⁺ or RNase H⁻forms) for AMV-RT showed the same activity in the continuous RT-PCR reaction.

In one step RT PCR, specific conditions have been developed for certain reverse transcriptases that are supportive of cDNA synthesis by RT and subsequent PCR amplification with a thermostable DNA polymerase such as Taq. The conditions described are specific for the specified reverse transcriptase. For example Lee and Rashtchian have described use of MMLV reverse transcriptase in conjunction with buffers containing MgSO₄, and Sellner et al have described use AMV type enzymes in SO₄-containing buffers for one-step RT PCR. Lee and Rashtchian (U.S. Pat. No. 6,495,350) have described use of Superscript II (an RNAse H− mutant of MMLV), providing a sensitive method for one-step RT PCR.

Despite this body of work, a significant difference in sensitivity and quantitation of different mRNAs is observed depending on which RT is used for reverse transcription. Some mRNAs appear to be preferred by one type of RT and others are preferred by other RTs. Therefore, there is presently no single methodology that is able to provide an accurate measure of different genes. The present invention provides a one-step RT-PCR method that uses two or more reverse transcriptases for providing accurate and unbiased quantitation of various mRNAs.

Example 1 shows quantitation of two different genes (as determined by the cycle threshold method) using 1-step RT PCR systems that employ MMLV or AMV and the combination of MMLV and AMV as the reverse transcriptase. The skilled artisan will appreciate that other RT enzymes can be used, for example, a combination of a single polypeptide RT, such as MMLV, and a multiple-subunit RT, such as AMV. As can be seen from the Example, when a single RT is used, GAPDH detection sensitivity is favored by MMLV by 4-10 fold, whereas β-actin is favored by AMV by 4-10 fold. However, when both enzymes are used in combination, the best sensitivities are observed regardless of which gene is being quantified. This invention accordingly provides methods and compositions for accurate and unbiased quantitation of mRNAs.

The present invention therefore provides compositions and methods useful for one-step/one-tube RT-PCR, preferably using two or more reverse transcriptases, or RNase H-deficient (“RNase H⁻”) derivatives thereof, in combination with one or more DNA polymerases. This invention is also directed to compositions and methods useful for one-step/one tube RT-PCR in the presence of buffer compositions (containing for example glutamate-containing molecules) that relieve the inhibition of PCR often observed when using compositions comprising two or more enzymes having reverse transcriptase activity.

In particular, the invention is directed to methods for amplifying a nucleic acid molecule comprising (a) mixing an RNA template with a composition comprising a first reverse transcriptase (for example a Moloney murine leukemia virus (M-MLV) reverse transcriptase) and a second reverse transcriptase (for example an Avian myoblastosis virus reverse transcriptase), in combination with one or more DNA polymerases and (b) incubating the mixture under conditions sufficient to amplify a DNA molecule complementary to all or a portion of the RNA template. In particular the invention contemplates use of reverse transcriptase mixtures containing a single polypeptide reverse transcriptase enzyme (for example M-MLV) and a reverse transcriptase enzyme containing two or more subunits, for example AMV or the HIV reverse transcriptase).

In a related aspect, the invention provides buffer compositions for use in RT-PCR methods that contain one or more components that suppress the inhibition of polymerase activity by the presence of one or more reverse transcriptase. Thus, for example, the present inventors have discovered that the presence of glutamate is effective for suppressing RT-mediated inhibition of polymerase activity. In particular, one or more glutamate-containing molecules, such as one or more glutamate-containing buffers, may be used in step (a) of the above-described methods, wherein the concentration of the one or more glutamate-containing molecules is about 1 mM to about 500 mM. In one embodiment of such methods, at least one DNA-directed DNA polymerase is used, for example, one, two, or three polymerases may be used, where one or more of the polymerases is a thermostable DNA polymerases. Suitable polymerases include, but are not limited to, Tne, Tma, Taq, Pfu, Tth, VENT, DEEPVENT, Pwo, and Tfl, or a mutant, variant or derivative thereof.

In other aspects of the invention, the DNA polymerases may comprise a first DNA polymerase having 3′ exonuclease activity, most preferably a DNA polymerase selected from the group consisting of Pfu, Pwo, DEEPVENT, VENT, Tne, Tma, Kod, and mutants, variants and derivatives thereof, and a second DNA polymerase having substantially reduced 3′ exonuclease activity, for example a DNA polymerase selected from the group consisting of Taq, Tfl, Tth, and mutants, variants and derivatives thereof. In other embodiments the reverse transcriptases used can be at least two of the following enzymes: Moloney Murine Leukemia Virus (M-MLV) RT, Avian Myeloblastosis Virus (AMV) RT, Thermoscript, RSV, Enhanced Avian RT, Sensiscript, OmniScript, Superscript I, Superscript II, Superscript III, Tth DNA polymerase, Human Immunodeficiency Virus (HIV), Avian Sarcoma-Leukosis Virus (ASLV) RT, Rous Sarcoma Virus (RSV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus UR2AV RT, Avian Sarcoma Virus Y73 Helper Virus YAV RT, Rous Associated Virus (RAV) RT, and Myeloblastosis Associated Virus (MAV) RT.

The compositions of the invention comprise, in addition to at least two reverse transcriptases and a DNA polymerase, one or more nucleotides, preferably deoxyribonucleoside triphosphates (most preferably dATP, dUTP, dTTP, dGTP or dCTP), dideoxyribonucleoside triphosphates (most preferably ddATP, ddUTP, ddGTP, ddTTP or ddCTP) or derivatives thereof. Such nucleotides may optionally be detectably labeled (e.g. with a radioactive or nonradioactive detectable label).

The compositions may also comprise one or more oligonucleotide primers suitable for priming first strand cDNA synthesis and for subsequent exponential amplification of the first strand cDNA, for example in a PCR. These primers may be, for example, oligo(dT) primers, random primers, arbitrary primers or target-specific primers. A target-specific primer may be a gene-specific primer.

The invention also provides methods in which the incubating step comprises (a) incubating the reaction mixture at a temperature (most preferably a temperature from about 25° C. to about 65° C.) and for a time sufficient to make a DNA molecule complementary to all or a portion of the RNA template; and (b) incubating the DNA molecule complementary to the RNA template at a temperature and for a time sufficient to amplify the DNA molecule, preferably via thermocycling, more preferably thermocycling comprising alternating heating and cooling of the mixture sufficient to amplify said DNA molecule, and most preferably thermocycling comprising alternating from a first temperature range of from about 90° C. to about 100° C., to a second temperature range of from about 40° C. to about 75° C., preferably from about 65° C. to about 75° C. In particularly preferred aspects of the invention, the thermocycling is performed greater than 10 times, more preferably greater than 20 times.

The invention is also directed to such methods wherein the amplification is not substantially inhibited by the presence of reverse transcriptase enzymes. That is, where the activity of the DNA polymerase enzyme is not substantially inhibited by the presence of reverse transcriptase enzymes.

Methods also are provided for amplifying a nucleic acid molecule comprising mixing an RNA template with a composition comprising a first reverse transcriptase enzyme in which the reverse transcriptase activity resides in a single polypeptide (for example Moloney murine leukemia virus (M-MLV) reverse transcriptase) and a second reverse transcriptase enzyme in which the reverse transcriptase activity resides in a dimeric or multimeric structure (for example AMV reverse transcriptase). Either enzyme, or both, may have reduced RNase H activity.

In the context of the present invention, a reverse transcriptase enzyme in which the reverse transcriptase activity resides in a dimeric or multimeric structure may also include engineered enzymes, where the individual protein chains that make up the dimeric or multimeric structure found in the enzyme as it occurs naturally have been linked to form a single polypeptide chain. The engineered single polypeptide chain retains the reverse transcriptase activity of the naturally-occurring dimeric or multimeric structure. See, for example, U.S. Patent Application 0020115147.

One or more DNA polymerases is added (most preferably selected from the group consisting of Tne, Tma, Taq, Pfu, Tth, VENT, DEEPVENT, Pwo, Tfl, and mutants, variants and derivatives thereof), as is one or more potassium-containing molecules, to form a reaction mixture. The reactants may be added in any order. The reaction mixture is incubated under conditions sufficient to amplify a DNA molecule complementary to all or a portion of the RNA template. In a related aspect, the invention is directed to such methods wherein one or more glutamate-containing molecules, such as one or more glutamate-containing buffers, is used in the reaction mixture.

The invention also provides methods for amplifying a nucleic acid molecule as described above comprising mixing the RNA template, the reverse transcriptase combination and the one or more DNA polymerases, where the unit ratio of reverse transcriptase to DNA polymerase is greater than about 3:2, and incubating the mixture under conditions sufficient to amplify a DNA molecule complementary to all or a portion of the RNA template.

The invention also is directed to compositions comprising a first reverse transcriptase enzyme in which the reverse transcriptase activity resides in a single polypeptide (for example Moloney murine leukemia virus (M-MLV) reverse transcriptase) and a second reverse transcriptase enzyme in which the reverse transcriptase activity resides in a dimeric or multimeric structure (for example AMV reverse transcriptase) and one or more DNA polymerases and one or more sulfur-containing molecules wherein the sulfur concentration is about 1 mM to about 500 mM), or combinations of one or more sulfur-containing molecules and one or more glutamate-containing molecules at the concentrations described above.

Compositions also are provided comprising a first reverse transcriptase enzyme in which the reverse transcriptase activity resides in a single polypeptide (for example Moloney murine leukemia virus (M-MLV) reverse transcriptase) and a second reverse transcriptase enzyme in which the reverse transcriptase activity resides in a dimeric or multimeric structure (for example AMV reverse transcriptase) with one or more DNA polymerases, one or more potassium-containing molecules and one or more sulfur-containing molecules or one or more glutamate-containing molecules (wherein the glutamate concentration is about 1 mM to about 500 mM), or combinations of one or more sulfur-containing molecules and one or more glutamate-containing molecules at the concentrations described above.

The glutamate-containing molecules advantageously are glutamate salts, for example, glutamate salts of organic bases, such as TRIS, TRICINE, BIS-TRICINE, MOPS, and/or TAPS glutamate, alkali metal salts such as sodium and/or potassium glutamates, alkaline earth metal salts such as calcium and/or magnesium glutamates, and other salts known in the art to be compatible with enzymatic activity.

The sulfur-containing molecules are preferably formulated into the present compositions in the form of one or more salts or buffers. Examples of suitable sulfur-containing salts according to the invention include, but are not limited to, ammonium sulfate, magnesium sulfate, manganese sulfate, potassium sulfate, sodium sulfate and the like. Examples of suitable sulfur-containing buffers according to the invention include, but are not limited to, TRIS-sulfate and other sulfuric acid-based buffers, as well as sulfonic acid-based buffers such as AMPSO (3-[(1,1-dimethyl-2-hydroxyethyl)amino]-2-hydroxy-propanesulfonic acid), BES (N,N-bis[2-hydroxyethyl]-2-aminomethanesulfonic acid), MOPS (3-N-morpholino)-propanesulfonic acid), MOPSO (3-N-morpholino)-2-hydroxypropanesulfonic acid, TES (2-{[tris-(hydroxymethyl)methyl]amino}ethanesulfonic acid), HEPES (N-2-hydroxyethylpiperazine-N′-2-ethansulfonic acid), HEPPS (N-2-hydroxyethylpiperazine-N′-3-propanesulfonic acid), HEPPSO (N-2-hydroxyethylpiperazine-N′-2-hydroxypropanesulfonic acid), TAPS(TES(3-{[tris-(hydroxymethyl)methyl]amino}propanesulfonic acid, CHES (2-(N-cyclo-hexylamino)ethanesulfonic acid), MES (2-N-morpholino)ethanesulfonic acid, PIPES (piperazine-N,N′-bis-2-ethanesulfonic acid), POPSO (piperazine-N,N′-bis[2-hydroxy]propanesulfonic acid), TAPS (N-tris[hydroxymethyl]methyl-3-aminopropanesulfonic acid), TAPSO (3-[N-tris{hydroxymethyl}methylamino]-2-hydroxypropanesulfonic acid), ACES (N-2-acetamide-2-aminoethane sulfonic acid), DIPSO (3-[N,N-bis(2-hydroxyethyl)amino]-2-hydroxypropanesulfonic acid), CAPSO (3-[cyclohexylamino]-2-hydroxy-1-propanesulfonic acid) and CAPS (3-[cyclohexylamino]propanesulfonic acid). Other sulfur-containing ionic salts and buffers, and other sulfur-containing molecules, suitable for use in the compositions of the invention will be apparent to one of ordinary skill in the art.

The potassium-containing molecules are preferably formulated into the present compositions in the form of one or more salts or buffers. Examples of suitable potassium salts according to the invention include, but are not limited to, potassium sulfate, potassium sulfite, potassium chloride, potassium nitrate, potassium acetate, monobasic and dibasic potassium phosphate and the like. Other potassium salts and buffers, and other potassium-containing molecules, suitable for use in the present compositions will be apparent to one of ordinary skill in the art.

The present invention, thus generally described, will be understood more readily by reference to the following examples, which are provided by way of illustration and are not intended to be limiting of the present invention.

### EXAMPLE 1

### Uniform cDNA Synthesis Efficiency Using a Reverse Transcriptase Mixture

This example demonstrates that nucleotide sequence affects the efficiency of first-strand synthesis by different reverse transcriptase enzymes. Use of a mixture of RTs from different sources overcomes sequence bias exhibited by a single RT and results in a more quantitative representation of RNA templates in the first-strand product.

Quantitative detection of human β-actin or glyceraldehyde-3-phosphate dehydrogenase (GAPDH) transcripts was carried out by real-time single-tube RT-PCR assay using varying amount of HeLa cell total RNA (log-fold serial dilutions, 1 pg to 10 ng input RNA) as template. 1-Step RT-PCRs were performed in 50-μL reaction volumes of 20 mM Tris-HCl (pH 8.4), 22.5 mM potassium chloride, 10 mM ammonium sulfate, 0.125M proline, 10% sucrose, 2.5 mM magnesium sulfate, 0.025% NP40, 0.025% Tween 20, 0.005% antifoam, 0.125×SYBR Green I, 10 nM fluorescein, 0.2 mM each dNTP, 2 μg/mL BSA, 1.25 units iTaq DNA polymerase (Bio-Rad Laboratories), and either 20 units MMLV reverse transcriptase (Invitrogen), 1 unit AMV reverse transcriptase (Seikagaku), or a combination of 10 units MMLV plus 0.5 units AMV, and 300 nM each primer. β-actin specific primers were as described by Xu et al. 2000, Focus 22:3-5 (forward primer: 5′-CCTGGCACCCAGCACAAT-3′; reverse primer: 5′-GGGCCGGACTCGTCATAC-3′). GAPDH specific primers were as described in the TaqMan GAPDH Control Reagents from Applied Biosystems, Inc., Foster City, Calif. (forward primer: 5′-GAAGGTGAAGGTCGGAGTC-3′; reverse primer: 5′-GAAGATGGTGATGGGATTTC-3′).

Reactions were assembled on ice in 96-well PCR plates. After addition of target RNA, plates were sealed with a heat-seal film and mixed. Following a brief centrifugation to collect reaction contents, reaction plates were transferred to a Bio-Rad iCycler that was equilibrated to 50° C. Reactions were incubated for 30 min at 50° C. followed by 5 min at 95 C to heat inactivate the reverse transcriptase. PCR amplification was carried through 45 cycles of: 10s, 95° C.; 20s, 60° C.; 30s, 68° C. Real-time optical monitoring was performed at the 68° C. extension step. Kinetic analysis and cycle threshold (Ct) determinations were performed using the iCycler iQ Optical System Software version 3.0 (Bio-Rad Laboratories). Results are summarized in Table 1.

Detection of GAPDH transcript occurred approximately 2 cycles earlier when MMLV RT was used for reverse transcription as compared to reactions using AMV RT. This was consistent across all levels of input RNA tested. Since PCR product approximately doubles with each PCR cycle, this indicates that ˜4 fold higher levels of GAPDH specific cDNA were obtained with MMLV RT. In contrast, detection of β-actin transcript occurred approximately 2.5 cycles earlier in AMV RT-PCRs compared to MMLV reactions. Again, this result was consistent across all levels of starting template RNA tested. Surprisingly, the use of a mixture of the two polymerases, under reaction conditions where each polymerase was active, did not result in interference between the two polymerases. Instead, Cts of detection for GAPDH or β-actin were comparable to those obtained with the optimal RT for each respective target sequence.

TABLE 1

| | Cts for GAPDH cDNA | | | Cts for β-actin cDNA | | |
|---|---|---|---|---|---|---|
| Input RNA | MMLV | AMV | MMLV | MMLV | AMV | MMLV |
| (pg) | RT | RT | & AMV | RT | RT | & AMV |
| 1 | 35.02 | 37.59 | 34.24 | 32.8 | 30.3 | 29.76 |
| 10 | 30.81 | 34.44 | 30.34 | 29.6 | 27.0 | 26.77 |
| 100 | 26.97 | 29.11 | 26.75 | 25.8 | 23.5 | 23.43 |
| 1000 | 23.2 | 25.16 | 22.58 | 22.4 | 20.0 | 20.02 |
| 100000 | 18.98 | 20.97 | 18.8 | 19.0 | 16.6 | 16.96 |
| Standard | −3.969 | −4.252 | −3.864 | −3.502 | −3.433 | −3.235 |
| Curve | | | | | | |
| Slope | | | | | | |
| Correlation | −1.0008 | −0.998 | −1.000 | −1.000 | −1.000 | −1.000 |
| Coefficient | | | | | | |

### EXAMPLE 2

### Biased cDNA Synthesis Efficiency Using a Reverse Transcriptase Mixture of Sensiscript and Omniscript

This example demonstrates that nucleotide sequence affects the efficiency of first-strand synthesis by different reverse transcriptase enzymes. Use of a mixture of OmniScript and SensiScript fails to overcome the sequence bias demonstrated in Example 1. Therefore, improved quantitative representation of RNA templates in one-step RT-PCR is not universal to any combination of reverse transcriptases.

Quantitative detection of human β-actin or glyceraldehyde-3-phosphate dehydrogenase (GAPDH) transcripts was carried out by real-time single-tube RT-PCR assay using varying amount of HeLa cell total RNA (log-fold serial dilutions, 5 pg to 50 ng input RNA) as template essentially as described in Example 1. Duplicate 1-Step RT-PCRs were performed in 25-μL reaction volumes for each input amount of RNA using components of the Quantitect SYBR Green RT-PCR kit (Qiagen, Inc.) according to the manufacturer's instructions. This kit utilizes a mixture of Sensiscript and Omniscript reverse transcriptase. Reactions also contained 10 nM fluorescein to permit normalization of fluorescent signal by the iCycler iQ Optical System Software. Control reactions using a mixture of MMLV and AMV RTs were carried out in 25-μL reaction volumes of 20 mM Tris-glutamate (pH 8.45), 10 mM L-glutamic acid, potassium salt, 10 mM ammonium sulfate, 0.125M proline, 5% sucrose, 5% glycerol, 2 mM magnesium sulfate, 0.025% NP40, 0.025% Tween 20, 0.005% antifoam, 0.2×SYBR Green I, 10 nM fluorescein, 0.2 mM each dNTP, 2.5 μg/mL BSA, 0.75 units iTaq DNA polymerase (Bio-Rad Laboratories), 5 units MMLV reverse transcriptase (Invitrogen), 0.25 unit AMV reverse transcriptase (Seikagaku). All reactions contained 300 nM each primer as described in Example 1.

Reactions were assembled on ice in 96-well PCR plates. After addition of target RNA, plates were sealed with a heat-seal film and mixed. Following a brief centrifugation to collect reaction contents, reaction plates were transferred to a Bio-Rad iCycler that was equilibrated to 50° C. Reactions were incubated for 30 min at 50° C. followed by 15 min at 95C to heat inactivate the reverse transcriptase. PCR amplification was carried through 45 cycles of: 10s, 95° C.; 20s, 60° C.; 30s, 72° C. Real-time optical monitoring was performed at the 72° C. extension step. Kinetic analysis and cycle threshold (Ct) determinations were performed using the iCycler iQ Optical System Software version 3.0 (Bio-Rad Laboratories). Results are summarized in Table 2.

In general, the Cts for detection of β-actin obtained with Sensiscript and Omniscript were comparable to those obtained using a mixture of AMV and MMLV RT at each input amount of RNA. However, the mixture of Sensiscript and OmniScript had difficulties amplifying the GAPDH sequence as Cts for detection of GAPDH at each RNA input amount were delayed relative to those obtained using MMLV and AMV RT. The disparity between the standard curve slope obtained with each RT mixture is also indicative of varying cDNA synthesis efficiency. These results are similar to those obtained for AMV alone as described in Example 1. Both Sensiscript and Omniscript are reported to be RNase H positive, heterodimeric reverse transcriptases and are therefore similar in molecular structure to AMV RT. It appears therefore that overcoming sequence bias in a one-step RT-PCR reaction may require the use of an MMLV-like reverse transcriptase in conjunction with an AMV-like reverse transcriptase.

TABLE 2

| | Average Cts for GAPDH | | Average Cts for β-actin | |
|---|---|---|---|---|
| | cDNA | | cDNA | |
| | Sensiscript | MMLV | SensiScript | MMLV |
| Input RNA | & | & | & | & |
| (pg) | Omniscript | AMV | Omniscript | AMV |
| 5 | 36.4 | 31.0 | 29.0 | 28.6 |
| 50 | 32.3 | 26.9 | 25.8 | 25.0 |
| 500 | 28.2 | 22.6 | 21.7 | 21.4 |
| 5000 | 21.0 | 17.8 | 18.3 | 18.4 |
| 500000 | 16.9 | 14.1 | 14.0 | 15.2 |
| Standard | −5.020 | −4.281 | −3.770 | −3.327 |
| Curve Slope | | | | |
| Correlation | −0.994 | −0.999 | −0.999 | −0.999 |
| Coefficient | | | | |

According to the methods of the invention, the reverse transcriptases, Taq DNA polymerase and buffers, dNTP's, cofactors and all other components for one step PCR can be mixed together in a variety of different concentrations to provide a ready to use mastermix.

Other preferred embodiments of the present invention will be apparent to one of ordinary skill in light of the foregoing description of the invention, and of the following claims.

What is claimed is:

1. A method for amplifying a nucleic acid molecule, said method comprising
incubating an RNA template with a composition comprising (a) a buffer, (b) two or more proteins having reverse transcriptase activity and (c) at least one DNA polymerase;
under conditions which substantially relieve reverse-transcriptase-mediated inhibition of DNA polymerase activity and which are sufficient to amplify a DNA molecule complementary to all or a portion of said RNA template.

2. A method according to claim 1, wherein said two or more proteins having reverse transcriptase activity comprise: a first reverse transcriptase enzyme in which the reverse transcriptase activity resides in a single polypeptide; and a second reverse transcriptase enzyme in which the reverse transcriptase activity resides in a dimeric or multimeric structure.

3. The method according to claim 2 wherein said first reverse transcriptase enzyme in which the reverse transcriptase activity resides in a single polypeptide is Moloney murine leukemia virus (M-MLV) reverse transcriptase or a derivative thereof having reduced RNAse H activity and said second reverse transcriptase enzyme in which the reverse transcriptase activity resides in a dimeric or multimeric structure is AMV reverse transcriptase or a derivative thereof having reduced RNAse H activity.

4. The method according to claim 1, wherein said composition comprises a first primer and a second primer,
wherein said first primer is suitable for facilitating synthesis of first strand cDNA from said RNA template, and wherein the combination of said first and said second primer is suitable for amplifying said first strand cDNA.

5. The method according to claim 1, wherein said buffer comprises an effective amount of at least one glutamate-containing compound.

6. The method according to claim 5, wherein the total glutamate concentration is about 1 mM to about 500 mM.

7. A method for accurately quantifying a nucleic acid molecule in an essentially sequence-independent manner, said method comprising
incubating an RNA template with a composition comprising (a) a buffer, (b) two or more proteins having reverse transcriptase activity, (c) at least one DNA polymerase, and (d) a first primer and a second primer,
wherein said first primer is suitable for facilitating synthesis of first strand cDNA from said RNA template, and wherein the combination of said first and said second primer is suitable for amplifying said first strand cDNA,
and wherein said incubation is under conditions which substantially relieve reverse-transcriptase-mediated inhibition of DNA polymerase activity and which are sufficient to amplify a DNA molecule complementary to all or a portion of said RNA template.

8. A method for the unbiased quantification of a nucleic acid molecule contained in a sample, said method comprising
incubating an RNA template with a composition comprising (a) a buffer, (b) two or more proteins having reverse transcriptase activity, (c) at least one DNA polymerase, and (d) a first primer and a second primer,
wherein said first primer is suitable for facilitating synthesis of first strand cDNA from said RNA template, and wherein the combination of said first and said second primer is suitable for amplifying said first strand cDNA,
and wherein said incubation is under conditions which substantially relieve reverse-transcriptase-mediated inhibition of DNA polymerase activity and which are sufficient to amplify a DNA molecule complementary to all or a portion of said RNA template.

9. A method according to claim 7, wherein said two or more proteins having reverse transcriptase activity comprise: a first reverse transcriptase enzyme in which the reverse transcriptase activity resides in a single polypeptide; and a second reverse transcriptase enzyme in which the reverse transcriptase activity resides in a dimeric or multimeric structure.

10. The method according to claim 9 wherein said first reverse transcriptase enzyme in which the reverse transcriptase activity resides in a single polypeptide is Moloney murine leukemia virus (M-MLV) reverse transcriptase or a derivative thereof having reduced RNAse H activity and said second reverse transcriptase enzyme in which the reverse transcriptase activity resides in a dimeric or multimeric structure is AMV reverse transcriptase or a derivative thereof having reduced RNAse H activity.

11. The method according to claim 8, wherein said two or more proteins having reverse transcriptase activity comprise: a first reverse transcriptase enzyme in which the reverse transcriptase activity resides in a single polypeptide; and a second reverse transcriptase enzyme in which the reverse transcriptase activity resides in a dimeric or multimeric structure.

12. The method according to claim 11 wherein said first reverse transcriptase enzyme in which the reverse transcriptase activity resides in a single polypeptide is Moloney murine leukemia virus (M-MLV) reverse transcriptase or a derivative thereof having reduced RNAse H activity and said second reverse transcriptase enzyme in which the reverse transcriptase activity resides in a dimeric or multimeric structure is AMV reverse transcriptase or a derivative thereof having reduced RNAse H activity.

13. The method according to claim 7, wherein said buffer comprises an effective amount of at least one glutamate-containing compound.

14. The method according to claim 8, wherein said buffer comprises an effective amount of at least one glutamate-containing compound.

15. The method according to claim 13, wherein said at least one glutamate compound is selected from the group consisting of glutamate salts of organic bases, alkali metal glutamate salts and alkaline earth metal glutamate salts.

16. The method according to claim 14, wherein said at least one glutamate compound is selected from the group consisting of glutamate salts of organic bases, alkali metal glutamate salts and alkaline earth metal glutamate salts.

17. The method according to claim 1, wherein said buffer further comprises an effective amount of an antifoam compound.

18. The method according to claim 5, wherein said buffer further comprises a sulfur-containing compound and a potassium-containing compound.

19. The method according to claim 1, wherein said DNA polymerase is a thermostable DNA polymerase.


SEQUENCE LISTING (4 sequences)

SEQ ID NO: 1 - DNA, 18 bp, Artificial Sequence
  Description of Artificial Sequence Primer
cctggcacccagcacaat

SEQ ID NO: 2 - DNA, 18 bp, Artificial Sequence
  Description of Artificial Sequence Primer
gggccggactcgtcatac

SEQ ID NO: 3 - DNA, 19 bp, Artificial Sequence
  Description of Artificial Sequence Primer
gaaggtgaaggtcggagtc

SEQ ID NO: 4 - DNA, 20 bp, Artificial Sequence
  Description of Artificial Sequence Primer
gaagatggtgatgggatttc